SEQUENCE LISTING (2 sequences)

SEQ ID NO: 1 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 2 - DNA, 1447 bp, Homo sapiens
  source 1..1447: mol_type = genomic DNA
gtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaaga

(19) United States
(12) Patent Application Publication
(10) Pub. No.: US 2024/0418727 A1
(43) Pub. Date: Dec. 19, 2024

(54) ACTIVATABLE NANOREPORTERS FOR REAL-TIME TRACKING OF MACROPHAGE PHENOTYPIC STATES ASSOCIATED WITH DISEASE PROGRESSION

(71) Applicant: University of Massachusetts, Boston, MA (US)

(72) Inventors: Ashish Kulkarni, Arlington, MA (US); Anujan Ramesh, Amherst, MA (US)

(21) Appl. No.: 18/743,008

(22) Filed: Jun. 13, 2024

Related U.S. Application Data
(60) Provisional application No. 63/507,801, filed on Jun. 13, 2023.

(51) Int. Cl. G01N 33/58; A61K 49/00; G01N 33/543; G01N 33/574

(52) U.S. Cl. CPC G01N 33/582; A61K 49/0052; G01N 33/5432; G01N 33/54346; G01N 33/57415; G01N 33/57484; G01N 2333/978

(57) ABSTRACT

An engineered a diagnostic lipid nanoparticle system that can provide early diagnosis of cancer.

### RELATED APPLICATIONS

This application claims the benefit of priority to U.S. Provisional Patent Application Ser. No. 63/507,801 entitled “ACTIVATABLE NANOREPORTERS FOR REAL-TIME TRACKING OF MACROPHAGE PHENOTYPIC STATES ASSOCIATED WITH DISEASE PROGRESSION,” filed Jun. 13, 2024, the disclosure of which is incorporated by reference herein in its entirety.

### INCORPORATION BY REFERENCE OF SEQUENCE LISTING

This application contains a Sequence Listing which has been submitted electronically in ST26 format and hereby incorporated by reference in its entirety. Said ST26 file, created on Aug. 5, 2024, is name 3724085US1.xml and is 4,651 bytes in size.

### BACKGROUND

Diagnosis of inflammatory diseases is characterized by identifying symptoms, biomarker validation, and imaging. However, these conventional techniques lack the sensitivities and specificities to detect disease early.

### SUMMARY

Provided herein is an engineered a diagnostic lipid nanoparticle system that can provide early diagnosis of cancer. In the tumor microenvironment, immune cells called macrophages acquire a tumor promoting characteristic known as an M2 phenotype and aid in tumor progression and metastasis by engaging in key molecular signaling pathways. In most pathological conditions macrophages exist as an inflammatory M1 phenotype, but as described earlier, in solid tumors such as triple-negative breast cancer (TNBC), the macrophages uniquely exist in an M2 phenotype. It has previously been identified that one of the main molecules responsible for macrophages acquiring an M2 phenotype is the enzyme Arginase-1. Therefore, it was hypothesized that longitudinal quantification of Arginase 1 levels in the tumor can predict disease outcome. To this end, using organic peptide synthesis chemistry, we successfully synthesized and characterized Arginase 1 sensing lipid nanoparticles that can selectively emit a strong fluorescent signal in the presence of M2 macrophages.

This is the first diagnostic nanoparticle system that can non-invasively monitor biomarkers that are indicative of tumor progression.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

Color photographs will be accepted if the conditions for accepting color drawings and black and white photographs have been satisfied. See 37CFR 1.84 (b)(2),

FIGS. 1A-1E: Schematics highlighting the mechanism of action of the M1 and M2 nanoreporters in various disease models: (a) Reaction schematics highlighting the mechanism of action of the M2 Nanoreporter. The Arginase 1 activatable probe remains “switched off” in steady-state conditions. However, the presence of Arginase 1 activates the fluorescent due to the cleavage of the quencher. (b) Reaction schematics highlighting the mechanism of action of the M1 Nanoreporter. The Nitric Oxide activatable probe remains “switched off” in steady-state conditions. However, the presence of Nitric Oxide activates the fluorescent due to the cleavage of the quencher due to hydrolysis. (c) Schematic representation of the 4T1 breast cancer model used to validate the efficacy of the M2 Nanoreporter in predicting the prognosis of breast cancer by detecting the presence of infiltrating M2 macrophages (d) Schematic representation of the local inflammation model where the M1 Nanoreporter has activated due to the nitric oxide present as a result subcutaneous inflammatory response that arose as a result of a local LPS administration (e) Schematic representation of muscle injury model to validate the M1+M2 Dual Nanoreporter. The injury was induced by intramuscular hind limb injection of turpentine oil. This was followed by an initial inflammatory response where M1 macrophages were detected at the site of inflammation, followed by a resolution phase signified by the infiltration of M2 macrophages involved in matrix regeneration and wound healing.

FIGS. 2A-21: Synthesis and Characterization of Nanoreporters: (a) Graph quantifying the relative time-dependent fluorescent intensity emitted by the M1 reporter probe when incubated with either lysate obtained from M1 lysate and M2 lysates. Statistical analysis was performed using 2 Way Anova with Sidak's multiple comparisons tests (b) Graph quantifying the fluorescent intensities emitted by various concentrations of the M2 reporter probe upon 24 h of incubation with either the M1 lysate or the M2 lysate. Data shown are mean±s.e.m. (n=3). Statistical analysis was performed using 2 Way Anova with Šídak's multiple comparisons test (c) Graph representing the stability of the M1 and M2 nano reporters. Stability is represented as an account of changes in the size of the nanoparticle system recorded for a period of 14 days in storage conditions (4° C.). Left Y axis corresponds to the M2 nanoreporter and the Right Y axis corresponds to the M1 nanoreporter. (d) Representative confocal image showing M1 and M2 Raw264.7 macrophages treated with the M2 nano reporter for a period of 4 h. Scale: 100 μm (e) Graph representing the quantification of fluorescent intensities obtained as a result of activation of the M2 nano reporter (green) in the presence of either M1 or M2 macrophages. Data shown are mean±s.c.m (n=3) (f) Representative confocal images showing M1 and M2 RAW264.7 macrophages treated with the M1 nano reporter for a period of 4 h. Scale: 100 μm (g) Graph representing the quantification of fluorescent intensities obtained as a result of activation of the M1 nano reporter (Red) in the presence of either M1 or M2 macrophages Data shown are mean±s.e.m. (n=3). (h) Representative Western blot images showing the expression of Arginase 1 and iNOS and Beta Actin in Raw264.7 macrophages that are either polarized to the M1 or the M2 phenotype. (i) Graph representing the quantification of protein expression in the western blot normalized to Beta Actin. Data shown are mean±s.e.m. (n=3). Statistical analysis was performed using Student's t-test for all data in the figure unless mentioned otherwise. * p<0.05, ** p<0.01. *** p<0.001. **** p<0.0001. * Compares the statistical significance between M2 macs and #compares the statistical significance between M1 macs.

FIGS. 3A-3J: Studies evaluating the efficacy of the M2 nanoreporter in a 4T1 breast cancer model: (a) Schematic illustration highlighting the mechanism of action of the M2 nano reporter. (b) Schematic illustration showing the dosage schedule of M2 Nano reporter administration. 4T1 Breast cancer cells were inoculated in the right flank of BALB/c mice. Dosing was started when tumors reached a volume of 50 mm³. The live mice images were captured at different time points using a Perkin Elmer IVIS Spectrum CT imaging system. (c) Graph showing the tumor progression profiles comparing control mice with mice treated with macrophage-depleting clodronate liposomes. Data shown are mean±s.e.m. (n=5). (d) Representative images of mice from different study groups imaged at different points. The tumors (dotted orange circle) show fluorescence activation due to the presence of Arginase 1 in the tumor microenvironment. (e) Graphs representing the quantification of fluorescent intensities obtained from different treatments at different time points. Data shown are mean±s.e.m. (n=5). Units of Average Radiant efficiency: [(p/see/cm²/sr)/(μW/cm²)]. (f) Representative fluorescent images of different organs obtained from mice subject to various treatments. (g) Graph showing the quantification fluorescent intensities emitted from different organs obtained from mice subject to different treatments. Data shown are mean±s.c.m. (n=5). (h) Representative confocal images showing the expression of F4/80 (red) in flash-frozen sections of tumors obtained from mice subject to different treatments. (i) Graph quantifying the number of F4/80+ve cells in tumors obtained from mice subject to different treatments (j) Graph showing RT PCR data quantifying the Arginase 1 levels in tumors obtained either from control mice or mice that have been subject to treatment with macrophage depleting clodronate liposomes. Data shown are mean #s.e.m. (n=3). Statistical analysis was performed using Student's t-test. * p<0.05. Data shown are mean±s.e.m. (n=3) Statistical analysis was performed using one-way ANOVA with Tukey posttest for all data in the figure unless mentioned otherwise. * p<0.05, ** p<0.01. *** p<0.001, **** p<0.0001.

FIGS. 4A-4G: Studies evaluating the efficacy of the M1 nanoreporter in a localized inflammation model: (a) Schematic representation of experimental design and the mechanism of action of the M1 Nano reporter in an animal model depicting local inflammation. (b) Schematic illustration showing the dosage schedule of M1 Nano reporter administration. A mixture of LPS and Matrigel was inoculated in the right flank of BALB/c mice. Dosing was started when one day b 110 after the Matrigel inoculation. The live mice images were captured at different time points using a Perkin Elmer IVIS Spectrum CT imaging system. (c) Representative images of mice from different study groups were imaged at different points. The Matrigel (dotted orange circle) shows fluorescence activation due to the presence of Nitric oxide released from macrophages inflammatory macrophages (d) Graph quantifying the relative fluorescent intensities emitted from the right flank of mice subject to various treatments. Units of Average Radiant efficiency: [(p/see/cm²/sr)/(μW/cm²)]. Data shown are mean±s.e.m. (n=5). (e) Representative fluorescent images of organs isolated from mice subject to different treatments. (f-g) Graphs quantifying the flow cytometry data that represents the expression of an M2 marker (CD206) and an M1 marker (CD80) on macrophages infiltrating the Matrigel. Data shown are mean±s.e.m. (n=3). Statistical 120 analysis was performed using one-way ANOVA with Tukey posttest for all data in the figure unless mentioned otherwise. * p<0.05. ** p<0.01. *** p<0.001, **** p<0.0001.

FIGS. 5A-51: Studies evaluating the efficacy of the M1+M2 Dual Nanoreporter in a muscle injury model: (a) Schematic representation of the experimental design and mechanism of action of the M1+M2 Dual nano reporter in a muscle injury model. (b) Schematic illustration showing the dosage schedule of M1+M2 Nano reporter. Muscle injury was induced by the administration of turpentine oil on the right hind limb muscle of BALB/c mice. Dosing was started 24 h after the injury was created. (c) Representative images of mice from different study groups were imaged at different points. The mice are imaged at the fluorescent window that corresponds to the excitation and emission of M1 nano reporters. The hind limbs (dotted orange circle) show fluorescence activation due to the presence of Nitric oxide released from inflammatory macrophages. (d) Representative images of mice from different study groups imaged at different points. The mice are imaged at the fluorescent window that corresponds to the excitation and emission of M2 nanoreporters. The hind limbs (dotted orange circle) show fluorescence activation due to the presence of Arginase 1 released from resolving macrophages. Units of Average Radiant efficiency: [(p/see/cm²/sr)/(μW/cm²)]. (e) Graphs quantifying the fluorescence emitted from the hind limbs of mouse subject to different treatments. Data shown are mean±s.e.m. (n=5). The fluorescent intensity was measured in the M1 Nanoreporter fluorescent window. (f) Graphs quantifying the fluorescence emitted from the hind limbs of mouse subject to different treatments. The fluorescent intensity was measured in the M2 Nano reporter fluorescent window. Data shown are mean±s.e.m. (n=5). (g) Representative H and E sections of hind limb muscles obtained from 140 mice included with a muscle injury at different time points. Scale: 1000 μm. Inserts represent infiltration of inflammatory monocytes denoted by the black arrow (h-i) Graphs quantifying the flow cytometry data that represents the expression of an M2 marker (CD206) and an M1 marker (CD80) on macrophages infiltrating the muscle. Data shown are mean±s.e.m. (n=3). Statistical analysis was performed using one-way ANOVA with Tukey posttest for all data in the figure unless mentioned otherwise. * p<0.05. ** p<0.01. *** p<0.001. **** p<0.0001.

### DESCRIPTION OF THE INVENTION

Provided herein is the detection of macrophage phenotypes, from inflammatory M1 to alternatively activated M2 macrophages, corresponding to the disease state can be used to predict the prognosis and diagnosis of various diseases. Activatable nanoreporters were engineered that can longitudinally detect the presence of the enzyme Arginase 1, a hallmark of M2 macrophages, and nitric oxide, a hallmark of M1 macrophages, in real-time in different disease models. Specifically, an M2 nanoreporter enabled the early imaging of the progression of breast cancer as predicted by selectively detecting M2 macrophages in tumors. Provided herein is a method to treat cancer, such as by chemotherapy, immunotherapy, surgery, and/or radiation. The M1 nanoreporter enabled real-time imaging of the subcutaneous inflammatory response that arose from a local LPS administration. Finally, the M1-M2 dual nanoreporter was evaluated in a muscle injury model, where an initial inflammatory response was monitored by imaging M1 macrophages at the site of inflammation, followed by a resolution phase monitored by the imaging of infiltrated M2 macrophages involved in matrix regeneration and wound healing. Provided herein is a method to 160 treat inflammation. This set of macrophage nanoreporters can be utilized for early diagnosis and longitudinal monitoring of inflammatory responses in various disease models.

Comprising eight exons, the ARG1 gene localizes at position 6q23.2 and codes for the manganese-dependent enzyme catalyzing the conversion of L-arginine into L-ornithine and urea (arginase 1; NM_000045.4). Arginase belongs to the ureohydrolase family of enzymes. Arginase catalyzes the fifth and final step in the urea cycle, a series of biochemical reactions in mammals during which the body disposes of harmful ammonia. Specifically, arginase converts L-arginine into L-ornithine and urea. Mammalian arginase is active as a trimer, but some bacterial arginases are hexameric. The enzyme requires a two-molecule metal cluster of manganese in order to maintain proper function. These Mn2+ ions coordinate with water, orienting and stabilizing the molecule and allowing water to act as a nucleophile and attack L-arginine, hydrolyzing it into ornithine and urea.

In most mammals, two isozymes of this enzyme exist; the first, Arginase I, functions in the urea cycle, and is located primarily in the cytoplasm of hepatocytes (liver cells). The second isozyme, Arginase II, has been implicated in the regulation of intracellular arginine/ornithine levels. It is located in mitochondria of several tissues in the body, with most abundance in the kidney and prostate. It may be found at lower levels in macrophages, lactating mammary glands, and brain. The second isozyme may be found in the absence of other urea cycle enzymes.

An exemplary human ARG1 protein sequence is as follows:

(SEQ ID NO: 1)
MSAKSRTIGIIGAPFSKGQPRGGVEEGPTVLRKAGLLEKLKEQEC
DVKDYGDLPFADIPNDSPFQIVKNPRSVGKASEQLAGKVAEVKKN
GRISLVLGGDHSLAIGSISGHARVHPDLGVIWVDAHTDINTPLIT
TSGNLHGQPVSFLLKELKGKIPDVPGFSWVTPCISAKDIVYIGLR
DVDPGEHYILKILGIKYFSMTEVDRLGIGKVMEETLSYLLGRKKR
PIHLSFDVDGLDPSFTPATGTPVVGGLTYREGLYITEEIYKTGLL
SGLDIMEVNPSLGKTPEEVTRTVNTAVAITLACFGLAREGNHKPI
DYLNPPK

or 80, 90, 95, 97, 100% identity thereto.

An exemplary human ARG1 coding sequence is as follows:

| | (SEQ ID NO: 2) |
|---|---|
| 1 | gtcactgagg gttgactgac tggagagctc |
| | aagtgcagca aagagaagtg tcagagcatg |
| 61 | agcgccaagt ccagaaccat agggattatt |
| | ggagctcctt tctcaaaggg acagccacga |
| 121 | ggaggggtgg aagaaggccc tacagtattg |
| | agaaaggctg gtctgcttga gaaacttaaa |
| 181 | gaacaagagt gtgatgtgaa ggattatggg |
| | gacctgccct ttgctgacat ccctaatgac |
| 241 | agtccctttc aaattgtgaa gaatccaagg |
| | tctgtgggaa aagcaagcga gcagctggct |
| 301 | ggcaaggtgg cagaagtcaa gaagaacgga |
| | agaatcagcc tggtgctggg cggagaccac |
| 361 | agtttggcaa ttggaagcat ctctggccat |
| | gccagggtcc accctgatct tggagtcatc |
| 421 | tcggtggatg ctcacactga tatcaacact |
| | ccactgacaa ccacaagtgg aaacttgcat |
| 481 | ggacaacctg tatctttcct cctgaaggaa |
| | ctaaaaggaa agattcccga tgtgccagga |
| 541 | ttctcctggg tgactccctg tatatctgcc |
| | aaggatattg tgtatattgg cttgagagac |
| 601 | gtggaccctg gggaacacta cattttgaaa |
| | actctaggca ttaaatactt ttcaatgact |
| 661 | gaagtggaca gactaggaat tggcaaggtg |
| | atggaagaaa cactcagcta tctactagga |
| 721 | agaaagaaaa ggccaattca tctaagtttt |
| | gatgttgacg gactggaccc atctttcaca |
| 781 | ccagctactg gcacaccagt cgtgggaggt |
| | ctgacataca gagaaggtct ctacatcaca |
| 841 | gaagaaatct acaaaacagg gctactctca |
| | ggattagata taatggaagt gaacccatcc |
| 901 | ctggggaaga caccagaaga agtaactcga |
| | acagtgaaca cagcagttgc aataaccttg |
| 961 | gcttgtttcg gacttgctcg ggagggtaat |
| | Cacaagccta ttgactacct taacccacct |
| 1021 | aagtaaatgt ggaaacatcc gatataaatc |
| | tcatagttaa tggcataatt agaaagctaa |
| 1081 | tcattttctt aagcatagag ttatccttct |
| | aaagacttgt tctttcagaa aaatgttttt |
| 1141 | ccaattagta taaactctac aaattccctc |
| | ttggtgtaaa attcaagatg tggaaattct |
| 1201 | aacttttttq aaatttaaaa gcttatattt |
| | tctaacttgg casaagactt atccttagaa |
| 1261 | agagaagtgt acattgattt ccaattaaaa |
| | atttgctggc attaaaaata agcacactta |
| 1321 | cataagcccc catacataga gtgggactct |
| | tggaatcagg agacaaagct accacatgtg |
| 1381 | gaaaggtact atgtgtccat gtcattcaaa |
| | aaatgtgatt ttttataata aactctttat |
| 1441 | aacaaga |

or 80, 90, 95, 97, 100% identity thereto.

### Example

### Introduction

Macrophages are a subset of immune cells that are characterized by their unique ability to perform a diverse set of functions. Macrophages display heterogeneous phenotypes that assist in executing such a vast array of functions ranging from phagocytosis to homeostasis. In a linear scale, macrophages can exist across a phenotype defined by an inflammatory phenotype on one end of the scale and an anti-inflammatory, disease-resolving phenotype on the other end of the scale.^{1,2} Macrophages orchestrate the development and progression of a variety of diseases such as infectious diseases, sepsis, inflammatory diseases (Rheumatoid arthritis, Fatty liver disease, irritable bowel syndrome) neurogenerative diseases, and cancers.³⁻⁶ The subsequent role played by macrophages in exacerbating disease outcomes is primarily dependent on their polarization state. For instance, chronic inflammatory diseases such as RA are characterized by infiltration of inflammatory cells, predominantly macrophages, that are in turn polarized to an M1 phenotype due to various known and unknown environmental cues resulting in an inflamed synovia.⁷ Activated M1 secrete inflammatory cytokines such as interleukin (IL)-1β, IL-6, and tumor necrosis factor (TNF)-α. These cytokines, in turn, promote inflammation by recruiting additional immune cells, that create a chronic inflammation condition.⁸⁻¹⁰ Similarly, most pathologies such as neurogenerative diseases, viral and bacterial infections, and sepsis are associated with an inflammatory macrophage response. However, the progression of a small subset of pathologies, mainly solid tumors is orchestrated by M2 macrophages. Infiltrating monocytes in the tumor microenvironment are differentiated and polarized to M2 macrophages owing to the cocktail of immunosuppressive cytokines in the tumor microenvironment. This results in the immunocompromised M2 macrophages that aid in tumor progression and eventual metastasis.¹¹

Inflammation plays a role in the pathogenesis of various diseases. In fact, inflammatory diseases and cancer taken together contribute to the majority of global mortality and morbidity which cause a significant economic burden on society.¹² Although there have been rapid strides in improving patient care, there are still no effective therapies available for chronic inflammatory diseases and solid metastatic cancers.¹³ Unlike acute inflammation, chronic inflammation often does not exhibit obvious signs and symptoms and is often neglected until the disease is clinically apparent. Developing tools that can effectively locate inflammation is critical for developing interventional strategies that can resolve inflammation.¹⁴ Current imaging and diagnostic techniques that monitor inflammation include the collection and analysis of biofluid and tissue biopsies. Biofluid analysis does not pinpoint the source of the inflammation, while tissue biopsies are extremely invasive. However, various nuclear imaging techniques are currently in development, that is specifically used to target macrophages in order to monitor inflammation.¹⁵ Small molecule ligands targeting the macrophage folate receptors have been investigated as a potential targeting agent to conjugate and deliver imaging agents, in addition, other targets such as the mannose receptor, oxidized LDL receptor (LOX-1), Translocator protein (TSPO) have also been investigated.¹⁶⁻¹⁹ Although these are novel targets, expression of surface receptors like Mannose and Folate have often been associated with M2 macrophages as well and might not be an exclusive biomarker inherent to M1 macrophages. In the case of solid tumors, early detection of the primary lesion is imperative in order to inhibit metastasis and reduce cancer mortality.¹⁵. Current clinical diagnostic strategies involving the measurement of endogenous biomarkers are not sensitive to detecting disease at a very early stage. Moreover, anatomical measurements of tumors using modalities such as MRI, and CT although can measure relatively small tumors, they may not be able to differentiate responders from non-responders, which is made possible due to molecular imaging.²¹⁻²³ There have been significant strides made in the field of early detection of diseases. Genetically encoded synthetic biomarkers reported by Gambhir and group, and pioneering work done by Bhatia and group on designing tumor penetrating nanosensors capable of detecting tumor protease levels are some works of notable mention.²⁴⁻²⁷ Provided herein is the use of macrophage-associated biomarkers in order to predict disease progression.

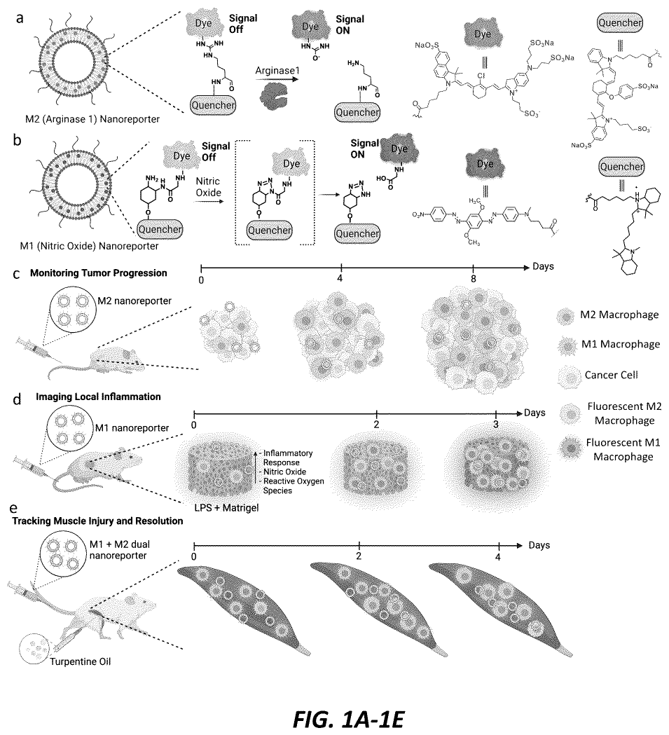

Leveraging the unique ability of macrophages to influence the pathogenesis of both inflammatory diseases and various cancers, it was hypothesized that designing reporter probes that can selectively monitor the inflammatory and the resolving macrophage phenotypes can be used as an effective strategy to predict the progression of the above-mentioned diseases. Currently, there are no such macrophage-based diagnostic strategies that can longitudinally predict disease outcomes. Inflammatory macrophages upregulate the enzyme iNOS (inducible Nitric Oxide Synthase) which in turn metabolizes the amino acid L arginine to nitric oxide. Nitric oxide is an important precursor that is responsible for the generation of reactive nitrogen species, which in turn plays a major role in aiding phagocytic cell death of incoming infections.²⁸ Resolving macrophages, on the other hand, are characterized by the enzyme Arginase 1, which hydrolyses L arginine to ornithine and urine. Sequestering L arginine not only makes it not available for NO synthesis, but the byproduct ornithine is also an important component of downstream pathways of polyamine and proline syntheses, which are important for cellular proliferation and tissue repair. Hence, it was hypothesized that designing activatable fluorescent probes that can detect inflammatory and resolving macrophage signatures by molecular imaging of Nitric Oxide and Arginase 1 respectively can visualize the polarization of the macrophages in either of their phenotypic states (FIG. 1A-1E).

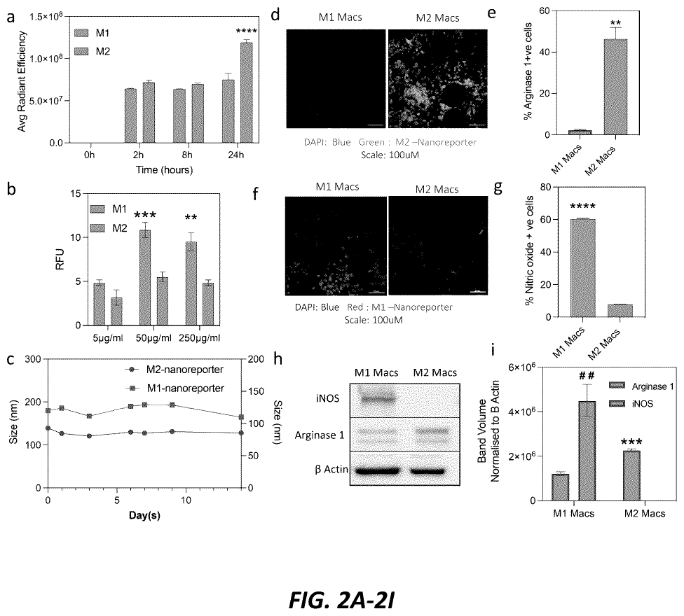

Fluorescent probes capable of imaging macrophage spectrums were synthesized to accomplish this goal. A highly reactive o-phenylenediamine molecule was used as the backbone to design the nitric oxide reactive probes. These O-phenylene diamine groups are highly reactive towards nitric oxide and in the presence of oxygen (O₂) undergo very selective and efficient benzotriazole ring formation. A series of chemical reactions on these o-phenylenediamine molecules allowed us to conjugate a visible range or a NIR (Near-infrared fluorophores) fluorophore and quencher on either side of this molecule. The proximity of the fluorophore results in the fluorescence being “turned off” state in the absence of a nitric oxide stimulus due to the fluorescence resonance energy transfer (FRET) phenomenon. Upon intracellular exposure to nitric oxide and in presence of oxygen, this newly engineered o-phenylenediamine moiety would undergo benzotriazole ring formation followed by hydrolysis to release the dye and quencher apart thereby emitting the fluorescent signal. Although in previous work, the efficacy of commercially available NO probes in detecting NO activity were reported, provided herein a more sensitive NO probe was developed that is modular in nature and can florescence in the more sensitive NIR window.²⁰ The efficacy of both the new NO probe and the Arginase 1 probe was evaluated under in vitro conditions was then evaluated. Briefly, RAW 264.7 macrophages were stimulated to the M1 after which the cells were lysed, and the lysate was incubated in varying concentrations of the M1 reporter probe. After an incubation time of 24 h, the fluorescence intensity was measured and plotted against an M2 control lysate that was obtained as a result of lysing RAW 264.7 macrophages stimulated to an M2 phenotype. As seen in FIG. 2A, there is a significant increase in the fluorescent signal at a concentration of >50 ug/ml as compared to the control, proving that the probe is indeed selective for M1 macrophages.

The arginase enzyme-responsive fluorescent peptide probe was synthesized using both solution-phase and solid-phase chemistry. The use of arginine amino acid as a central node in the peptide sequence allowed us to deploy the fluorophore and a quencher on either side of the active site of the arginase I enzyme. This probe was also designed on the same FRET principle wherein these probe remains in “off” if fluorophore and quencher are in close proximity. The cleavage of the arginase 1 functional site on the peptide probe would release the dye and quencher aside to emit the fluorescence signal. To validate the specificity of probe activation, M1 and M2 Raw 264.7 macrophage lysates were prepared as per the brief protocol mentioned earlier. The Arginase 1 reporter probe was incubated at a concentration of 100 ug/ml and the fluorescence signal emitted was measured at regular time intervals. As seen in FIG. 2B, there is a significant increase in the fluorescent signal emitted in the lysate obtained from M2 macrophages as compared to the lysate obtained from M1 macrophages, highlighting the specificity of the arginase probe.

Systemic delivery of probes that get selectively activated in the presence of disease by a process mediated by enzymatic cleavage of probes can possibly achieve enhanced signal-to-noise ratios and high sensitivities as compared to current disease evaluation techniques. However, these strategies are limited by a number of factors such as lack of biocompatibility of the probe, premature clearance, and ability of the probes to get distributed to the disease site. Therefore, to address these challenges, a strategy was implemented, wherein the Nitric Oxide and Arginase 1 reporter probes were encapsulated in liposomal nanoparticle systems to form the M1 and M2 nanoreporter respectively. The nanoreporter systems were synthesized using a thin-film hydration technique facilitated by the self-assembly of Phosphatidylcholine, DSPE PEG Amine, cholesterol, and the respective reporter probes. Phosphatidylcholine was chosen for its biocompatible nature, DSPE PEG Amine was used as a “Pegylating” agent, and cholesterol was used to stabilize the lipid bilayer. The resulting M1 and M2 nano reporters had a size of 118.54+7.23 nm and 128.8+5.15 nm respectively and zeta potential of and 9.26t1.57 mV and 9.348+0.59 mV respectively. The stability of the nanoparticle systems was evaluated by measuring the size and surface charge at regular intervals over a course of 14 days. As seen in FIG. 2C, minimal changes in size and surface charge were observed over the due course of storage, proving that the nanoparticles are indeed stable under storage conditions. Furthermore, extensive characterization of this liposomal nanoparticle system in previous studies shows the stability of the system in physiological conditions of 37° C. and 10% serum as well.^{20.29.30} The encapsulation efficiency of the nanoparticle systems was also evaluated and was found to be 70%.

Upon evaluating the encapsulating efficacy, the mechanism of release of the probes from the nanoparticle system was investigated. The liposomal nanoparticle system was previously reported by the group to preferentially release its cargo in a sustained manner upon internalization into the macrophage compartment.^{29.30} In order to validate the previously reported mechanism, M1+M2 nanoreporters were encapsulated in the nanoparticle system and incubated them in either a macrophage lysate or PBS and estimated the drug release profiles as a function of time. As shown in, the probes are released in a sustained manner in the group incubated with the macrophage lysate but not in the PBS group. This could be due to the fact that the acidic nature of the macrophage lysate due to the presence of lysosomal components facilitates the disruption of the nanoparticles to release the probes in a sustained manner.

To evaluate the imaging functions of the nanoreporters. RAW 264.7 macrophages were seeded and either polarized to an M1 phenotype or an M2 phenotype. Two sets of experiments were performed, in the first set, the macrophages were incubated with the M1 nanoreporter, and in the other set of experiments, the cells were incubated with the M2 nanoreporter. Upon incubation of the nanoreporter, the cells were fixed and counterstained with DAPI and imaged. As shown in FIG. 2D-G, it can be observed that the M2 nanoreporter gets selectively activated in the presence of M2 macrophages, and alternatively, the M1 nanoreporter gets selectively activated in the presence of M1 macrophages. In order to further validate the hypothesis that the enzyme Arginase 1 and the small molecule Nitric Oxide are specific molecules associated with the M2 and the M1 macrophage phenotypes respectively, a western blot was performed quantifying the expression levels of these molecules in M1 and M2 macrophages. As shown in FIG. 2H-I, it is indeed evident that Nitric Oxide and Arginase 1 are specifically overexpressed in M1 and M2 macrophages respectively and are suitable targets for molecular imaging of macrophage phenotypes. Upon successfully synthesizing the M1 and M2 nanoreporters and evaluating their sensitivities and specificities in detecting their respective macrophage phenotypes, the efficiency of these probes in predicting the progression of various disease states was evaluated.

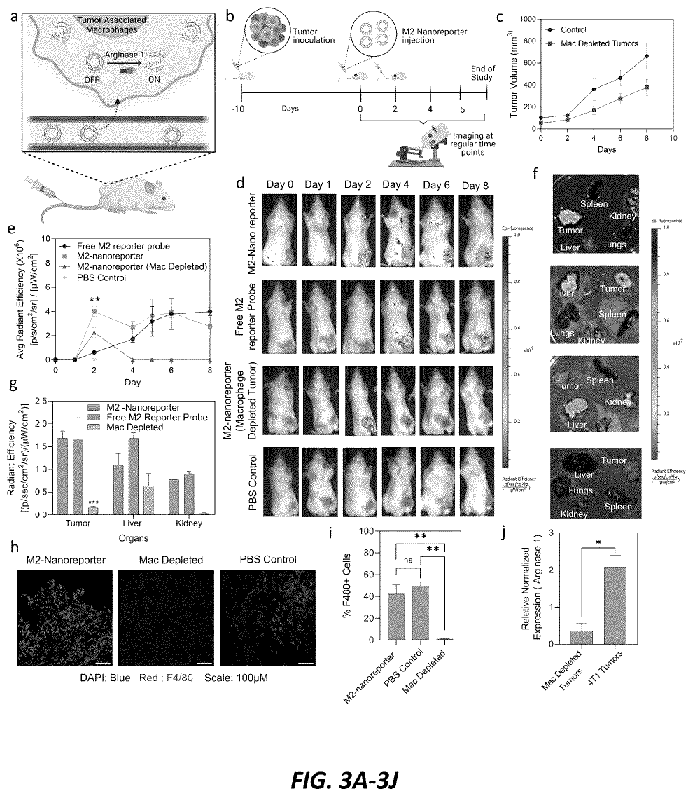

Triple-negative breast cancer (TNBC) is characterized by its distinct lack of expression of receptors that are hard to treat with conventional therapies.³¹ The aggressive nature of TNBC, coupled with poor therapeutic response rates poses an urgent need to engineer effective strategies for disease management. Early detection of metastases and migration, especially in aggressive cancers like TNBCs are avenues of research that can hold the key to reducing cancer mortality.³² Detection of endogenous biomarkers such as circulating tumor DNA, miRNAs, and exosomes, although have been used to predict the progression of disease in many cases, they are still unreliable as a single modality and need other diagnostic markers in combination for them to predict to reliably relay information with regards to disease outcome. In addition, metastatic and primary tumors from the same patients vary at a genetic level, along with a lack of sensitivity to differentiate endogenous DNA biomarkers from tumor-related DNA are some major limitations.^{33,34} Due to the overlying disadvantages posed by the current diagnostic techniques mentioned above, there is a need for an alternative strategy that can longitudinally monitor the progression of TNBC sensitively and selectively. Metastasis, invasion, and drug resistance associated with TNBC have been strongly linked to the intratumor infiltration of tumor-associated macrophages (TAMs).^{35,36} In the tumor microenvironment, these TAMs acquire a tumor-promoting characteristic known as an M2 phenotype and aid in tumor progression and metastasis by engaging in key molecular signaling pathways. Leveraging this unique phenomenon, it was hypothesized that diagnostic sensors that can specifically and selectively detect the presence of M2 macrophages can predict TNBC progression and metastasis at a very early stage by using macrophage polarization as a predictive diagnostic marker. (FIG. 3A).

In order to determine whether M2 nanoreporters can detect tumors in vivo, a syngeneic triple-negative 4T1 breast cancer mouse model was used where 4T1 cells (1×10⁶ cells) were injected into the right flank subcutaneously and the tumors were allowed to develop. On the 7th day, when the tumors reached a size of 50 mm³, mice were randomized into groups of n=5. However, in one of the groups, TAMs were depleted by administering chlodronate-containing liposomes. This group serves as a “no macrophage” control in order to reinforce the fact that M2 nano reporters are specific in detecting only M2 macrophages. M2 nanoreporters were injected intravenously at a concentration of 2.5 mg/kg in the group containing 4T1 tumor-bearing mice and the group containing 4T1 tumor-bearing mice with depleted macrophages. Free M2 reporter probe was administered at equimolar concentrations in tumor-bearing mice, while a control group that was not administered with any probes was used for subtraction of the background signal. The fluorescence spectra of the dye attached to the M2 nano reporter had a maximum excitation peak of 774 nm and an emission of 789 nm. Mice were administered a total of 2 doses of the probes split over alternate days, with the day of the first dose injection being day 0. (FIG. 3B) The M2 nano reporter can detect tumors as early as 48 h when the tumor size is <100 mm³ as evidenced by the appearance of a bright fluorescent signal that is 2-fold higher than the free probe group. As previously hypothesized, no signal emitted from macrophage-depleted tumor-bearing mice upon macrophage depletion further validates the specificity of the nanoreporter. However, one has to note that clodronate-liposomes deplete macrophages for 24-72 hours, warranting a second dose of injection to achieve depletion. This can be observed by the appearance of a faint fluorescent signal in the macrophage-depleted control group on day 2 due to the rebound of macrophages at the tumor site, which subsequently disappeared upon a second dose of clodronate-liposome injection. In mice administered with the free probe, a delayed response is observed, where tumors are detected on day 4 instead of day 2 as seen in the nano reporter group. Mice were euthanized at the end of day 8, where a relatively strong signal was still observed in both the free probe group as well as the nano reporter group. Endpoint ex vivo imaging of the tumors reveals that not only does a strong positive signal corresponding to Arginase 1 persist in tumors obtained from mice administered with the nano reporter and the free probe, but it is also evident that there are relatively low nonspecific signals that are emitted from organs associated with the Reticuloendothelial system (RES), primarily the liver, lungs, and kidney, with the liver being the highest. This result is expected given that the tissue-resident macrophages in the liver contribute to nonspecific signals arising due to the presence of Arginase 1. (Figure C-G). In order to further prove the hypothesis that M2 nano reporters specifically detect M2 macrophages as a result of longitudinal sensing of Arginase 1, it was shown that tumors obtained from mice treated with chlodronate-containing liposomes are indeed devoid of macrophages as evidenced by the immunofluorescent images of tumors isolated from different groups (FIG. 3H-1) Additionally, qPCR analysis of whole tumors for Arginase 1 expression shows that tumor devoid of macrophages indeed have low Arginase I expression profiles as compared to the tumors obtained from the macrophage competent mice leading to lower fluorescent signals. (FIG. 3J) Furthermore, to ensure that the signal that arises as a result of the administration of the M2 nano reporter is a result of specific cleavage of the probe by Arginase 1, a control experiment was performed where mice inoculated with 4T1 TNBC cells were administered the M1 nano reporter, and the mice were imaged using the IVIS after 24 h of injection. The M2 nano reporter is indeed specific in predicting the M2 macrophage phenotype, while the M1 nano reporter is associated with no significant non-specific signal in the M2 animal model.

Inflammation is a naturally occurring phenomenon in the body that is indicative of the body's immune response in fighting illness and infection. Acute inflammatory conditions arise due to injuries in the body, bacterial and viral infections and are typically characterized by a severe and quick inflammatory response followed by resolution and eventual tissue repair.³⁷ Acute inflammations are characterized by the infiltration of pro-inflammatory immune cells, typically monocyte, macrophages, and T cells working together to eliminate the impending cause of inflammation. This is subsequently followed by a wound healing and resolving phase signified by a phenotypic switch of immune cells, particularly macrophages, to a resolving phenotype that supports wound healing and tissue regrowth to achieve homeostasis.³⁸⁻⁴⁰ Contrastingly, Chronic inflammatory conditions are characterized by a dysregulation of the immune response that is characterized by a hyperactive inflammatory T cell and macrophage response. The overstimulated adaptive immune response is characterized by an inflammatory innate immune response, which leads to a perpetual state of inflammation that never heals. The exact reason for chronic inflammation is unknown, with several factors such as social environment and lifestyle factors, and dysregulated autoimmunity influencing a person's ability to fight the inflammation. Today, chronic inflammatory diseases are one of the leading causes of death, contributing to about 50% of all deaths worldwide and substantial efforts are being taken to find strategies for early diagnosis, prevention, and treatment of chronic inflammatory diseases.⁴¹⁻⁴³

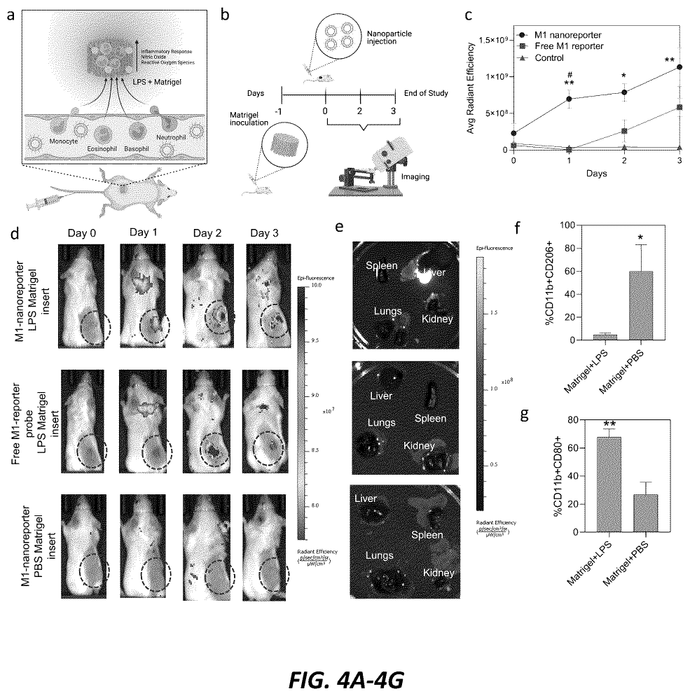

However, the fact that chronic inflammatory diseases often exhibit no symptoms at the onset of disease represents a major diagnostic challenge. In addition to this, currently, there are no effective tests to assess patients for chronic inflammation. Serum protein electrophoresis and the highly sensitive C-reactive protein assays are non-reliant as they are highly nonspecific. Detection of serum levels of pro-inflammatory markers can provide some evidence, but these tests are not standardized and cannot pinpoint the exact tissue-level source of inflammation.⁴²⁻⁴⁴ Therefore, in order to bridge some of these limitations and answer questions that are beyond current diagnostic, there is a need to image biochemical processes that are key contributors of inflammation in a minimally invasive and site-specific manner. Molecular imaging of inflammation, hence, has the potential to transform the diagnosis of chronic inflammatory diseases. As mentioned earlier, macrophages are important mediators of inflammation and play a crucial role in the progression of chronic inflammation. The macrophages recruited to the site of inflammation acquire an inflammatory phenotype and secrete inflammatory mediators such as iNOS, ILIBeta, IL6, and TNF alpha. The presence of these inflammatory mediators at the site of inflammation further exacerbates the injury leading to a state of persistent inflammation.⁹ Therefore, we hypothesized that molecular imaging of nitric oxide at the site of inflammation might be predictive of inflammation and this information would be relayed in a site-specific manner. Although the ability of the liposomal nanoparticle system to preferentially accumulate in the tumor site was previously evaluated.³⁰ the biodistribution of the M1 nano reporter compared to the distribution of the free probe was evaluated. Upon 24 h of administration, there is a relatively higher accumulation of the M1 probe at the site of the Matrigel when delivered via the liposomal system as compared to the free probe administration. To evaluate the efficacy of the nitric oxide sensing M1 nanoreporter in reporting the presence of M1 macrophages, an animal model was established that has been induced with a local inflammation that can redirect macrophages to the inflammatory site so that they can subsequently get differentiated to an M1 phenotype. In the study performed by Taddio et al, the group established a local inflammation mouse model where mice were subcutaneously (s.c.) inoculated with Matrigel containing LPS. This served as a scaffold for the infiltration of inflammatory macrophages at the Matrigel site.⁴⁵ As shown in the schematics in FIG. 4A-B, BALB/C mice were either inoculated with an LPS/Matrigel gel insert or a control containing a PBS/Matrigel insert. The mice were then randomly sorted and assigned to different treatments. After an inoculation period of 24 h, The M1-nano reporter (Dose: 2 mg/kg) was administered to two groups of mice, a group inoculated with the LPS insert and another group containing the control PBS insert. Whereas the free M1 reporter probe was administered to the group only with the LPS insert. The day of administration of the probes was day 0. Mice were imaged at regular intervals using the In vivo imaging system at the excitation wavelength corresponding to the fluorophore of the M1 reporter (Ex: 651 nm and nm: 670 nm). As seen in FIG. 4C-D, the mice inoculated with the LPS/Matrigel insert are compared with mice inoculated with the PBS/Matrigel insert with both groups getting two doses of equimolar concentration of the M1 nano reporter. There is a clear and apparent appearance of a fluorescent signal that is 5-fold higher when compared to other groups at 24 h after administration in the LPS group, whereas, this is absent in the control group, hence pointing to the fact that the signal is specific in detecting an inflammatory response. It is important to note that this signal was site-specific and was present only at the site where the Matrigel insert was injected, barring minimal nonspecific auto-fluorescent signal emitting from the surface of the skin of the mouse.⁴⁶ Moreover, the fluorescent signal intensity was sustained till day 3 in mice administered with the M1 nano reporter, whereas not only is the fluorescent signal in the free M1-reporter probe treated delayed and appears only on day 2, but it is not sustained as well, disappearing at day 3.

Additionally, there are no apparent fluorescent signals emitted from organs isolated from mice subject to different treatments, highlighting the specificity of the M1 Nano reporter in detecting only detecting Nitric oxide. (FIG. 4E) In order to further reinforce the fact that the M1 nano reporter are specific in detecting only M1 macrophages, macrophages were depleted by administering chlodronate containing liposomes. This group serves as a “no macrophage” control. There is no apparent fluorescent signal that arises from the Matrigel inoculation site when the mice are imaged after 24 h of nano reporter administration emphasizing the fact that the M1 nano reporter specifically gets activated as a result of the presence of inflammatory M1 macrophages. In order to confirm that the signal that is obtained in the mice with the LPS/Matrigel insert is indeed due to the infiltration of inflammatory macrophages, the macrophage population infiltrating the Matrigel was quantified. As shown in FIG. 4F-G, macrophages infiltrating the LPS insert are characterized by a high expression of CD80 and low expression of CD206 signifying an M1 phenotype, whereas macrophages infiltrating the PBS group are characterized by a CD80 lo and CD206 hi signifying a more M2 like phenotype. Furthermore, to ensure that the signal that arises a result of administration of the M1 nano reporter is a result of specific cleavage of the probe by Nitric Oxide, a control experiment was performed where mice inoculated with LPS Matrigel were administered the M2 nano reporter, and the mice were imaged using the IVIS after 24 h of injection. No significant nonspecific signal was observed at the site of the Matrigel proving that the M1 nano reporter is indeed specific in predicting the M1 macrophage phenotype. The M1 nano reporter is indeed specific in predicting the M1 macrophage phenotype, while the M2 nano reporter is associated with no significant non-specific signal in the M1 animal model.

Monitoring the progression of chronic inflammation can provide an opportunity to strategize clinical interventions that can lead to eventual resolution and eventual wound healing. Herein it has been clearly demonstrated that dynamic molecular imaging of the inflammatory site for the presence of inflammatory macrophages can help establish the presence of chronic inflammatory conditions. Additionally, it is also believed that longitudinal imaging of resolving macrophages could enable direct monitoring of the action of different treatment modalities aimed at resolving inflammation and promoting wound healing. It has already been established that the resolution phase of wound healing is characterized by the presence of resolving macrophages that upregulate the enzyme Arginase 1. Therefore, it was hypothesized that designing a reporter system that can monitor both the inflammatory phase as well the resolving phase of macrophages will not only provide spatial information on the location of the inflammation but can monitor the resolution of the inflammation in response to various therapies. This signals a paradigm shift in the diagnosis and treatment of chronic inflammatory diseases. To this end, an M1+M2 dual nano reporter was designed that co-encapsulates both the M1 reporter probe and the M2 reporter probe and can be used as a sensor to longitudinally monitor both the inflammatory and resolution spectrums.

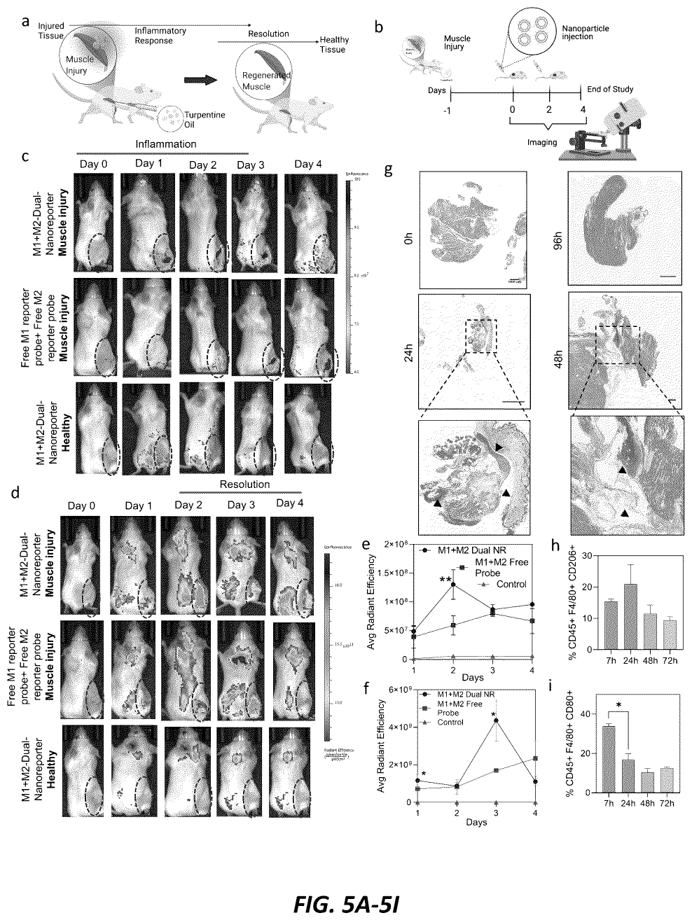

To establish an animal model that can exhibit a distinct inflammatory phase followed by a resolution phase, we referred to the work done by Aalipour et al.²⁷ To test their diagnostic cell-based Arginase 1 sensor, they induced a muscle injury model, where turpentine oil was injected in the hind limb to initiate the inflammation. This inflammatory phase was gradually replaced by a resolution phase, characterized by infiltrating M2 macrophages overexpressing Arginase 1. (FIG. 5A) Additionally, a control group was established where mice were administered with a PBS injection instead of turpentine oil. M1+M2 dual nano reporters were administered to the group with the muscle injury and the healthy mice with no injury whereas, a combination of a free M1 reporter probe and an M2 reporter probe was co-administered to mice induced with a hind leg muscle injury. The mice were administered a total of two doses, where the injection of the first dose (injected 1 day after inducing the injury) was Day 0. (FIG. 5B) As seen in FIG. 5C-F, in injured mice administered with the M1+M2 Dual-nano reporter day I was characterized by a marked increase in the fluorescent intensity emitted in the excitation channel corresponding to the M1 probe, whereas this no fluorescence is observed in the channel corresponding to the M2 reporter probe. It is taken into account that injured mice administered the free probes were also marked by the presence of a strong fluorescent intensity emitted from the hind limbs, but a delayed response was observed where the signal was observed on day 2 instead. The healthy mice showed baseline levels of fluorescence. In the fluorescent window corresponding to the M2 nano reporter, it was observed that injured mice administered with the M1+M2 Dual-nano reporters and the free probe combination are characterized by the appearance of a positive signal on day 2 that persists through day 4, the eventual endpoint. However, healthy mice that were administered with the probe only emitted baseline levels of fluorescence. These results taken together indicate that the M1+M2 dual nano reporter is indeed capable of sensing the acute inflammatory phase that follows a tissue injury, as well the resolution phase characterized by the increased infiltration of wound resolving M2 macrophages. Furthermore, it is evident that there is no apparent fluorescent signal that are emitted from organs isolated from mice from different treatment groups highlighting the specificity of the dual M1+M2 Nano reporter in detecting Nitric Oxide and Arginase 1 respectively.

To further validate these observations H and E staining was performed on muscle tissue obtained from mice that are induced with the hind leg injury. It can be seen that the 24 h time point is characterized by an increased presence of infiltrating inflammatory cells that eventually get cleared and give rise to a resolved injury at the end of 5 days (FIG. 5G). These results were further validated by flow cytometry performed on the muscle tissue to evaluate the phenotype of the infiltrating macrophages. Flow cytometry results concur with the observations seen in FIG. 5H-I, where indeed at 7 h we see inflammatory macrophages characterized by their CD80hi and CD206 low phenotype, which gradually turn to a wound-healing phenotype by gradually downregulating the co-stimulatory cytokines CD80 through the progression of time.

In conclusion, provided herein is the synthesis and characterization of novel M1 and M2 nanoreporters that can specifically and selectively detect the presence of nitric oxide, a small molecule associated with M1-like macrophages, and Arginase 1, an enzyme associated with M2-like macrophages respectively. The M1 and M2 nanoreporters were found to be stable in extended storage and had a size of 118.5417.23 nm and 128.815.15 nm respectively and zeta potential of and 9.26+1.57 mV and 9.348+0.59 mV. In vitro, experiments demonstrate the capability of the nano reporters in predicting the M1-like and M2-like macrophage phenotypic states as a result of longitudinal sensing of Nitric oxide and Arginase I respectively. The application of the nanoprobes was further validated in detecting various diseases by evaluating the (1) M2 nano reporter in a breast cancer mouse model, where the onset and prognosis of breast cancer was predicted by detecting M2 macrophages. (2) The M1 nano reporter, which was evaluated in a local inflammation model, where we detected the subcutaneous inflammatory response that arose as a result of a local LPS administration. (3) An M1-M2 dual nano reporter that is capable of detecting macrophages on both side of the spectrum. The efficacy of the M1-M2 dual reporter was evaluated in a muscle injury model, where an injury was induced by intramuscular hind limb injection of turpentine oil. This was followed by an initial inflammatory response where M1 macrophages were detected at the site of inflammation, followed by a resolution phase signified by the infiltration of M2 macrophages involved in matrix regeneration and wound healing. As discussed earlier, macrophages play a unique and crucial role in the pathogenesis of various inflammatory diseases. In these studies, we have successfully demonstrated that longitudinal imaging of different macrophage phenotypes can be used as a predictive indicator of progression of various diseases.

### BIBLIOGRAPHY

1. M. Orecchioni, Y. Ghosheh, A. B. Pramod, K. Ley, Frontiers in immunology, 2019, 10, 1084.
2. C. D. Mills, Frontiers in immunology, 2015, 6, 212.
3. J. A. Ardura, G. Rackov, E. Izquierdo, V. Alonso, A. R. Gortazar, M. M. Escribese, Front Pharmacol, 2019, 10, 1255.
4. S. R. Nielsen, M. C. Schmid, Mediators Inflamm, 2017, 2017, 9624760.
5. S. I. Grivennikov, F. R. Greten, M. Karin, Cell, 2010, 140, 883.
6. K. Prame Kumar, A. J. Nicholls, C. H. Y. Wong, Cell Tissue Res, 2018, 371, 551.
7. R. W. Kinne, R. Brauer, B. Stuhlmuller, E. Palombo-Kinne, G. R. Burmester, Arthritis Res, 2000, 2, 189.
8. S. Gordon, L. Martinez-Pomares, Pflugers Arch, 2017, 469, 365.
9. N. Fujiwara, K. Kobayashi, Curr Drug Targets Inflamm Allergy, 2005, 4, 281.
10. Y. C. Liu, X. B. Zou, Y. F. Chai, Y. M. Yao, Int J Biol Sci, 2014, 10, 520.
11. A. Sica, M. Erreni, P. Allavena, C. Porta, Cellular and molecular life sciences: CMLS, 2015, 72, 4111.
12. C. Zhang, M. Yang, A. C. Ericsson, Frontiers in immunology, 2021, 12, 620510.
13. I. B. McInnes, E. M. Gravallese, Nature reviews. Immunology, 2021, 21, 680.
14. C. H. Liu, et al., FASEB J. 2019, 33, 13085.
15. A. L. Fisse, J. Motte, T. Gruter, M. Sgodzai, K. Pitarokoili, R. Gold, Neurol Res Pract, 2020, 2, 42.
16. Y. S. Yi, Immune network, 2016. 16, 337.
17. K. Movahedi, et al., Cancer research, 2012, 72, 4165.
18. R. Rupprecht. V. Papadopoulos, G. Rammes, T. C. Baghai, J. Fan, N. Akula, G. Groyer, D. Adams, M. Schumacher, Nature reviews. Drug discovery, 2010, 9, 971.
19. S. Ishino, et al., Journal of nuclear medicine: official publication, Society of Nuclear Medicine, 2008, 49, 1677.
20. A. Ramesh, S. Kumar, A. Brouillard, D. Nandi, A. Kulkarni, Adv Mater, 2020, 32, e2000648.
21. F. Aydin, L. Dertsiz, E. S. Budak, A. Yildiz, G. Ozbilim, F. Gungor, Diagn Interv Radiol, 2013, 19, 271.
22. M. E. Seaman, G. Contino, N. Bardeesy, K. A. Kelly, Expert Rev Mol Med, 2010, 12, e20.
23. A. E. Marciscano, D. L. J. Thorek, Adv Radiat Oncol, 2018, 3, 534.
24. J. S. Dudani, A. D. Warren, S. N. Bhatia, Annual Review of Cancer Biology, 2018, 2, 353.
25. J. S. Dudani, M. Ibrahim, J. Kirkpatrick, A. D. Warren, S. N. Bhatia, Proceedings of the National Academy of Sciences of the United States of America, 2018, 115, 8954.
26. G. A. Kwong, S. Ghosh, L. Gamboa, C. Patriotis, S. Srivastava, S. N. Bhatia, Nature reviews. Cancer, 2021, 21, 655.
27. A. Aalipour, et al., Nature biotechnology, 2019, 37, 531.
28 J. E. Albina, J. S. Reichner, Cancer Metastasis Rev, 1998, 17. 39.
29. A. Ramesh, S. Kumar, D. Nandi, A. Kulkarni, Adv Mater, 2019, 37, e1904364.
30. A. Kulkarni, et al., Nat Biomed Eng, 2018, 2, 589.
31. L. Yin, J. J. Duan, X. W. Bian, S. C. Yu, Breast cancer research: BCR, 2020, 22, 61.
32. G. Bianchini, J. M. Balko, I. A. Mayer, M. E. Sanders, L. Gianni, Nature reviews. Clinical oncology, 2016, 13, 674.
33. S. S. Hori, S. S. Gambhir, Science translational medicine, 2011, 3, 109ra116.
34. H. Schwarzenbach, D. S. Hoon, K. Pantel, Nature reviews. Cancer, 2011, 17, 426.
35. L. Cassetta, J. W. Pollard, Cell Res, 2017, 27, 963.
36. H. Jeong, I. Hwang, S. H. Kang, H. C. Shin, S. Y. Kwon, J Breast Cancer, 2019, 22, 38.
37. L. Chen, H. Deng, H. Cui, J. Fang, Z. Zuo, J. Deng, Y. Li, X. Wang, L. Zhao, Oncotarget, 2018, 9, 7204.
38. L. C. Davies, S. J. Jenkins, J. E. Allen, P. R. Taylor, Nature immunology, 2013, 14, 986.
39. A. C. Gonzalez, T. F. Costa, Z. A. Andrade, A. R. Medrado, An Bras Dermatol, 2016, 91, 614.
40. T. A. Wynn, K. M. Vannella, Inununity, 2016, 44. 450.
41. V. Jairath, B. G. Feagan, Lancet Gastroenterol Hepatol, 2020, 5, 2.
42. J. L. Schultze, S. consortium, P. Rosenstiel, Immunity, 2018, 48, 608.
43. D. Furman, et al., Nature medicine, 2019, 25, 1822.
44. H. Maillard-Lefebvre, E. Boulanger, M. Daroux, C. Gaxatte, B. I. Hudson, M. Lambert, Rheumatology (Oxford), 2009, 48, 1190.
45. M. F. Taddio, et al., Molecular imaging and biology: MIB: the official publication of the Academy of Molecular Imaging, 2021, 23, 196.
46. T. Troy, D. Jekic-McMullen, L. Sambucetti, B. Rice, Mol Imaging, 2004, 3, 9.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

While the present disclosure has been described with reference to the specific embodiments thereof, it should be understood by those skilled in the art that various changes may be made and equivalents may be substituted without departing from the true spirit and scope of the present disclosure. In addition, many modifications may be made to adapt a particular situation, material, composition of matter, process, process step or steps, to the objective, spirit and scope of the present disclosure. All such modifications are intended to be within the scope of the claims appended hereto.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed is:

1. A M1 reporter probe comprising a reactive o-phenylenediamine molecule conjugated to a fluorophore and a quencher.

2. The M1 reporter probe of claim 1, wherein the reporter probe specifically detects the presence of nitric oxide.

3. M2 reporter probe comprising an arginase 1 enzyme conjugated to a fluorophore and a quencher.

4. The M2 reporter probe of claim 3, wherein the reporter probe specifically detects the presence of Arginase 1.

5. The reporter probe of claim 1, wherein the reporter probe is encapsulated in liposomal nanoparticle.

6. The reporter probe of claim 3, wherein the reporter probe is encapsulated in liposomal nanoparticle.

7. A nanoreporter comprising an M1 report prove and an M2 reporter probe encapsulated in a liposomal nanoparticle.

8. The nanoreporter of claim 7, wherein the reporter probes detects the M1-like and M2-like macrophage phenotypic states as a result of longitudinal sensing of nitric oxide and arginase 1 respectively.

9. A method to detect disease, injury and/or inflammation comprising contacting a cell with or administering the M1 reporter probe of claim 1.

10. A method to detect disease, injury and/or inflammation comprising contacting a cell with or administering the M2 reporter probe of claim 3.

11. The M1 reporter probe of claim 1 for use in longitudinal imaging of different macrophage phenotypes to indicate progression of disease/efficacy of treatment.

12. The M2 reporter probe of claim 3 for use in longitudinal imaging of different macrophage phenotypes to indicate progression of disease/efficacy of treatment.

13. The method of claim 9, wherein the disease is cancer.

14. The method of claim 10, wherein the disease is cancer.

15. The method of claim 13, wherein the cancer is breast cancer.

16. The method of claim 14, wherein the cancer is breast cancer.

17. The method of claim 9, wherein the injury is muscle injury.

18. The method of claim 10, wherein the injury is muscle injury.

19. A method to visualize the polarization of the macrophages in either of their phenotypic states comprising contacting a cell with one more M1 and/or M2 reporter probes to detect and resolve macrophage signatures by molecular imaging of nitric oxide and arginase I.

20. A method to make a M1 or M2 report probe/nanoreporter as described herein.
